(19) United States
(12) Patent Application Publication
(10) Pub. No.: US 2022/0224843 A1
(43) Pub. Date: Jul. 14, 2022

(54) SMART EYEGLASSES FOR IMPROVING VISUAL IMPAIRMENT AND FACILITATING VISUAL FIELD TESTS

(71) Applicant: MGK Innovations, LLC, Las Vegas, NV (US)

(72) Inventors: Glenn Adamousky, Edison, NJ (US); James Tharayil, Edison, NJ (US)

(21) Appl. No.: 17/643,900

(22) Filed: Dec. 13, 2021

Related U.S. Application Data
(60) Provisional application No. 63/199,596, filed on Jan. 11, 2021.

(51) Int. Cl. H04N 5/262; G06F 1/16; G06F 3/01; H04N 5/232; G02C 7/02; G02C 11/00; A61B 3/00; A61B 3/024; B64C 39/02

(52) U.S. Cl. CPC H04N 5/2628; G06F 1/163; G06F 3/012; H04N 5/23229; G02C 7/02; G06F 3/167; A61B 3/005; A61B 3/0033; A61B 3/0091; A61B 3/024; B64C 39/024; G02C 11/10

(57) ABSTRACT

A pair of eyeglasses including right and left temples, one or more lenses/displays, one or more gyroscopes, one or more sensors configured to determine the position of a wearer's eyes, one or more cameras, one or more processors running software configured to receive input in the form of video content from said one or more cameras and output manipulated video content to said one or more lenses/displays for viewing by a wearer thereof. The eyeglasses may be used to provide visual field tests to wearers in an actual real-life environment rather than an office setting.

### CROSS-REFERENCE

This application claims priority to U.S. Patent Application No. 63/199,596 filed Jan. 11, 2021 and is incorporated herein by reference for all purposes.

### FIELD OF THE INVENTION

The embodiments of the present invention relate generally to eyeglasses incorporating software and/or hardware elements to improve a wearer's impaired vision.

### BACKGROUND

Visual impairment, also known as vision impairment or vision loss, is a decreased ability to see to a degree that causes problems not fixable by usual means, such as conventional eyeglasses or contacts. The causes of visual impairment are many including, but not limited to, refractive errors, cataracts, glaucoma, stroke, premature birth and trauma.

Peripheral vision loss (PVL) occurs when a person cannot see objects unless the objects are directly in front of the person. This is also known as tunnel vision. Loss of side vision can create obstacles in daily life, often impacting one's overall orientation, how they get around and how well they see at night.

Thus, it would be advantageous to develop a solution for visual impairment involving improved eyeglasses incorporating software and/or hardware elements for allowing a wearer to benefit from improved vision.

### SUMMARY

The embodiments of the present invention generally involve a pair of eyeglasses including right and left temples, one or more lenses/displays, one or more gyroscopes, one or more sensors configured to determine the position of a wearer's eyes, two or more cameras, one or more processors running software configured to receive input in the form of video content from said two or more cameras and output manipulated video content to said one or more lenses/displays for viewing by a wearer thereof.

In another embodiment, a portion of the eyeglasses including at least one of the two or more cameras may be disengaged to serve as a drone. In this manner, the user may be provided with aerial views of his or her surroundings via the lenses/displays. In this embodiment, the eyeglasses include one or more wireless receivers, transmitters and/or transceivers that provide a communication link between the drone portion and the eyeglass portion being worn.

In another embodiment, the eyeglasses are configured to receive digital communications such as emails, text messages and the like. The messages may be displayed on the lenses/displays.

In another embodiment, the functionality of the eyeglasses is controlled by voice commands. In such an embodiment, the eyeglasses include voice recognition software running on the processor.

Other variations, embodiments and features of the present invention will become evident from the following detailed description, drawings and claims.

### BRIEF DESCRIPTION OF THE DRAWINGS

FIG. 1 illustrates a perspective view of an exemplary pair of eyeglasses according to the embodiments of the present invention;

FIG. 2 illustrates a front view of an exemplary pair of eyeglasses according to the embodiments of the present invention;

FIG. 3 illustrates a top view of an exemplary pair of eyeglasses according to the embodiments of the present invention;

FIG. 4 illustrates a first side view of an exemplary pair of eyeglasses according to the embodiments of the present invention;

FIG. 5 illustrates a first side view of an exemplary pair of eyeglasses according to the embodiments of the present invention;

FIG. 6 illustrates a remote control device according to the embodiments of the present invention;

FIG. 7 illustrates a block diagram of hardware/software associated with the eyeglasses according to the embodiments of the present invention;

FIG. 8 illustrates a diagram of a portion of the eyeglasses acting as a drone according to the embodiments of the present invention; and

FIG. 9 illustrates a display depicting a visual field test of the type that may be facilitated by the embodiments of the present invention.

### DETAILED DESCRIPTION

For the purposes of promoting an understanding of the principles in accordance with the embodiments of the present invention, reference will now be made to the embodiments illustrated in the drawings and specific language will be used to describe the same. It will nevertheless be understood that no limitation of the scope of the invention is thereby intended. Any alterations and further modifications of the inventive feature illustrated herein, and any additional applications of the principles of the invention as illustrated herein, which would normally occur to one skilled in the relevant art and having possession of this disclosure, are to be considered within the scope of the invention claimed.

Those skilled in the art will recognize that the virtual, digital and online embodiments of the present invention involve both hardware and software elements which portions are described below in such detail required to construct and operate a game method and system according to the embodiments of the present invention.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.), or an embodiment combining software and hardware. Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), and optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied thereon, for example, in baseband or as part of a carrier wave. Such a propagated signal may take any variety of forms, including, but not limited to, electromagnetic, optical, or any suitable combination thereof. A computer readable signal medium may be any computer readable medium that is not a computer readable storage medium and that can communicate, propagate, or transport a program for use by or in conjunction with an instruction execution system, apparatus, or device.

Program code embodied on a computer readable medium may be transmitted using any appropriate medium, including but not limited to wireless, wireline, optical fiber cable, RF and the like, or any suitable combination of the foregoing.

Computer program code for carrying out operations for aspects of the present invention may be written in any combination of one or more programming languages, including an object oriented programming language such as Java, Smalltalk, C++ or the like or conventional procedural programming languages, such as the “C” programming language, AJAX, PHP, HTML, XHTML, Ruby, CSS or similar programming languages. The programming code may be configured in an application, an operating system, as part of a system firmware, or any suitable combination thereof. The programming code may execute entirely on the user's computer, partly on the user's computer, as a standalone software package, partly on the user's computer and partly on a remote computer or entirely on a remote computer or server as in a client/server relationship sometimes known as cloud computing. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram.

The computer program instructions may also be loaded onto a computer, other programmable data processing apparatus, or other devices to cause a series of operational steps to be performed on the computer, other programmable apparatus or other devices to produce a computer-implemented process such that the instructions which execute on the computer or other programmable apparatus provide processes for implementing the functions/acts specified in the flowchart and/or block diagrams.

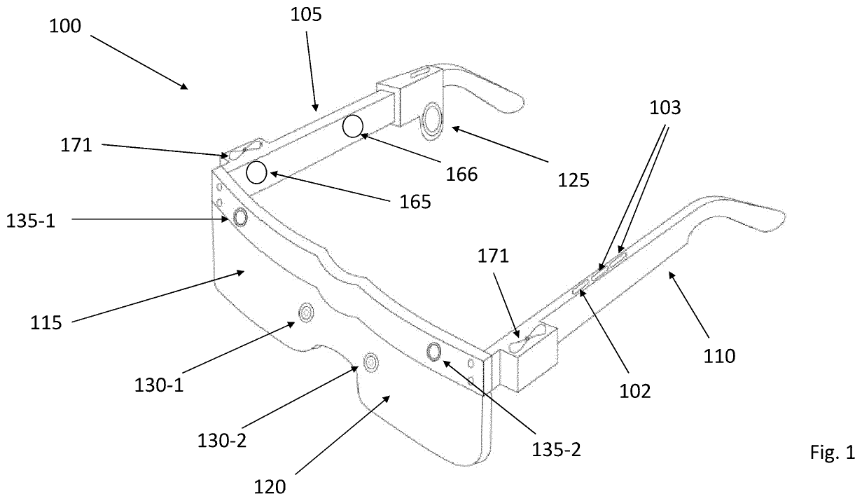

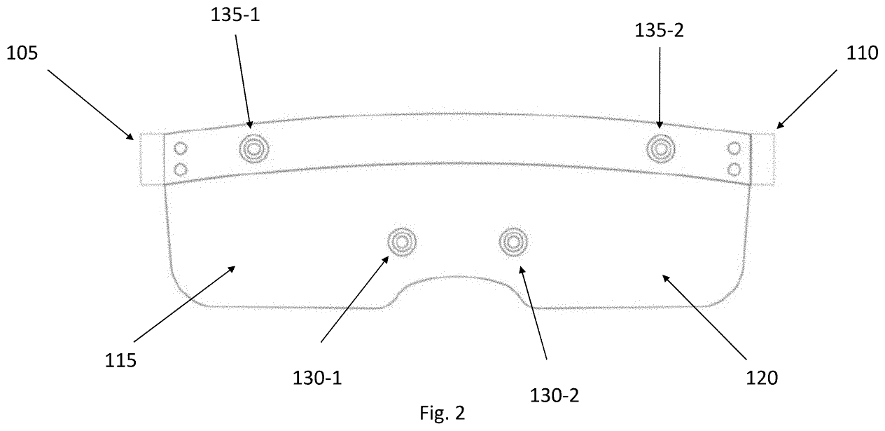

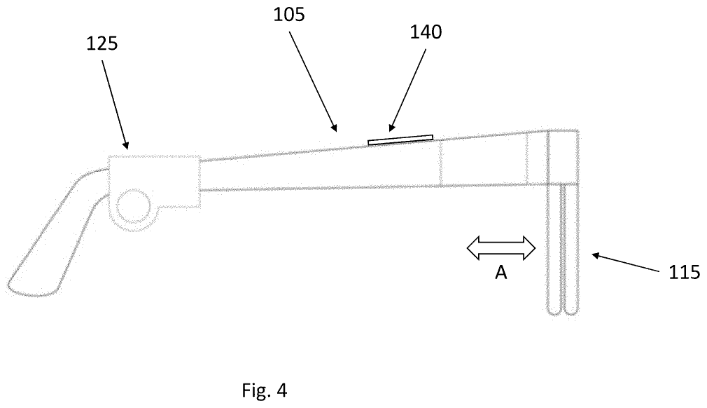

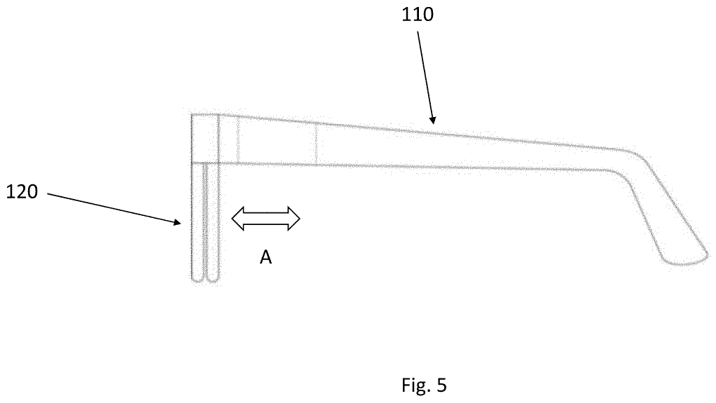

FIGS. 1-5 show various views of a pair of eyeglasses 100 according to the embodiments of the present invention. Those skilled in the art will recognize that the design and appearance of the eyeglasses 100 are exemplary only such that other designs and appearances are within the embodiments of the present invention. In general terms, the eyeglasses 100 include a right temple 105, left temple 110, right lens/display 115, left lens/display 120, gyroscope 125, sensors 130-1, 130-2 configured to determine the position of a wearer's eyes, cameras 135-1, 135-2, a processor 200 running software 201 configured to receive input in the form of video content from the cameras 135-1, 135-2 and output manipulated video content to the right lens/display 115, left lens/display 120 for viewing by a wearer thereof. The right lens/display 115 and left lens/display 120 may be separate articles or part of a single article (as shown in FIGS. 1 and 2). While two cameras 135-1, 135-2 are shown, one camera, with multiple lenses, may be used as well as more than two cameras.

A primary objective, although by no means the only objective, of the eyeglasses detailed herein is to improve visual impairment of the wearer. In general, video content captured by the cameras 135-1, 135-2 is run through software running on the processor and presented on the right lens/display 115 and left lens/display 120. In one embodiment, the software manipulates the video content relative to the wearer's particular visual impairment. For example, if the visual impairment is the loss of peripheral vision, the software may manage the video content to address the peripheral vision loss by reducing the size of the video content field so that more video content is displayed in the wearer's direct vision field on the right lens/display 115 and left lens/display 120. In this manner, the wearer will be presented with certain peripheral vision objects in their direct field of vision.

In another embodiment, the presentation of the video content on the right lens/display 115 and left lens/display 120 is altered based on data collected by gyroscope 125 and sensors 130-1, 130-2. Sensors 130-1, 130-2 may be cameras, scanners, biometric readers, or other video image capturing devices directed at the wearer's eyes. The objective of the sensors 130-1, 130-2 is to capture in real-time the gaze (i.e., pupil position) of the wearer. Knowing the gaze of the wearer allows the processor to alter the presentation of the video content to reflect the gaze of the wearer. For example, if the gaze of the wearer's eyes is detected by the sensors 130-1, 130-2 to be to the right, the presentation of the video content may be skewed by the processor to the right on the right lens/display 115 and left lens/display 120. Similarly, the gyroscope 125 may be used to determine a wearer's head position to better present the video content on the right lens/display 115 and left lens/display 120.

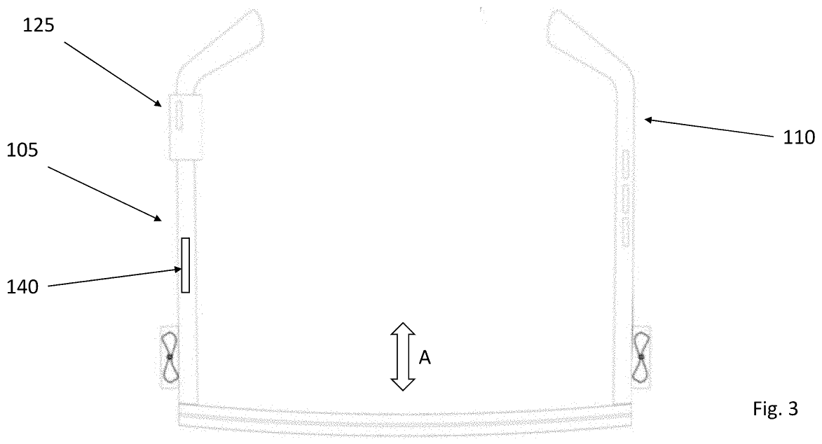

As shown by arrow A in FIGS. 3-5, the right lens/display 115 and left lens/display 120 may be moved, manually or automatically, to modify the distance between the wearer's eyes and the right lens/display 115 and left lens/display 120. In automated embodiments, the processor may control the movement based on input from the gyroscope 125 and sensors 130-1, 130-2. Alternatively, the wearer may provide instructions to move to the right lens/display 115 and left lens/display 120 as desired. The instructions, as detailed below, may be input via voice commands, via a local interface (e.g., toggle 140 or slider) or remote interface 145 (e.g., remote control). Automated movement of the right lens/display 115 and left lens/display 120 may be controlled by one or more motors 142, servos or similar devices integrated into the eyeglasses 100.

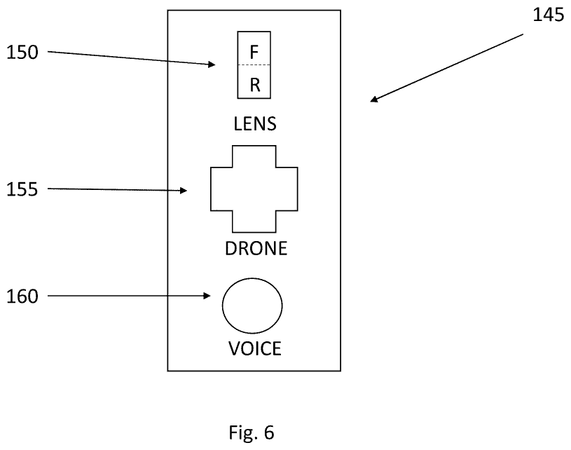

FIG. 6 shows an exemplary remote-control device 145 for operating certain functionality associated with the eyeglasses 100. The remote control device 145, as shown, includes an input 150 for moving the right lens/display 115 and left lens/display 120 forward (F) and rearward (R), a drone control input 155 and voice common input microphone 160. Receiver 162 is configured to receive wireless communications, including voice and drone commands, transmitted by the remote control device 145. The voice commands may also be directly received via microphone 165 on the eyeglasses 100. In one embodiment, the remote control device 145 communicates with the eyeglasses 100 via a wireless communication link 165 (e.g., Bluetooth, wi-fi, cellular, etc.). The remote control device 145 (and drone 170) may also be communicatively connected to the eyeglasses 100 via a wire (not shown).

In another embodiment, the eyeglasses 100 are configured to communicatively link to smart devices 180 such as smart phones and tablets. In one embodiment, the smart devices 180 transmit information such as emails and text messages to the eyeglasses 100 for presentation on the right lens/display 115 and left lens/display 120. Calls may also be directed to the eyeglasses 100, via cellular, WiFi or Bluetooth connections with the smart device, such that the wearer may use microphone 165 and speaker 166 to complete the call. Receiver 162 or another receiver may collect the smart device signals while transmitter 163 may send wearer voice transmissions.

The smart devices 180 may also be used to control the functionality of the eyeglasses 100. In such an embodiment, a software application (“App”) downloaded onto the smart device 180 provides a virtual user interface to the wearer. The virtual interface operates like the remote control device 145 interface shown in FIG. 6.

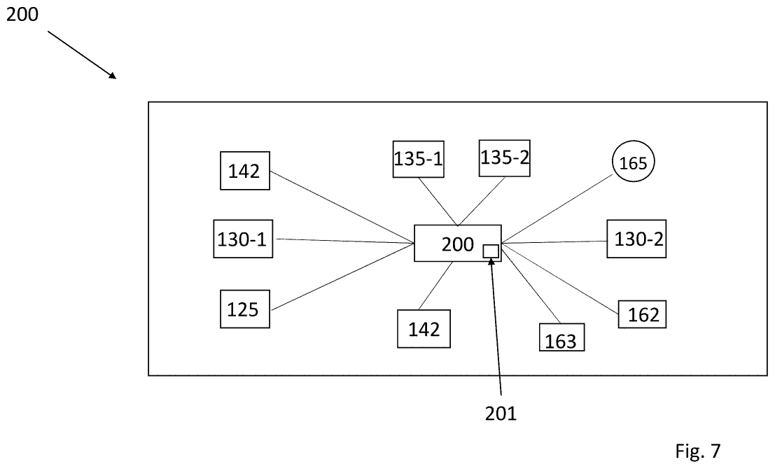

FIG. 7 shows a block diagram 205 of hardware/software associated with the eyeglasses 100 according to the embodiments of the present invention. A processor 200 manages functionality associated with the eyeglasses 100. In one embodiment, the processor 200 is the Quad-core ARM Cortex-A57 MPCore made by Nvidia Jetson Nano SoM including WiFi and Bluetooth modules. The processor 200 communicates and/or instructs gyroscope 125, sensors 130-1, 130-2, cameras 135-1, 135-2, motor 142, receiver 162, transmitter 163, microphone 165 and speaker 166. In one embodiment, the cameras 135-1, 135-2 incorporate Qualcomm video modules using the QCS605 processor. Although block diagram 205 shows that the processor 200 communicates and/or instructs all components, in other embodiments, other processors and controllers may be used such that a single central processor does not manage all functionality.

In one embodiment, the one or more processors may incorporate artificial intelligence (AI) capabilities to assist with managing the functionality of the eyeglasses 100. AI may be used, for example, to utilize historical data to control the automatic position of the right lens/display 115 and left lens/display 120 to maximize the viewing experience of the wearer.

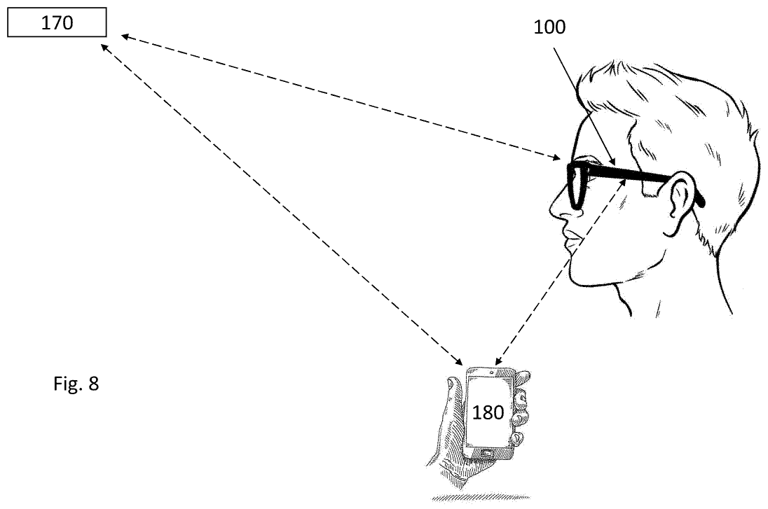

FIG. 8 shows a diagram 300 of a portion of the eyeglasses 100 acting as a drone 170 according to the embodiments of the present invention. When forming a portion of the eyeglasses 100, propellers/rotors 171 provide lift to the drone 170. In one embodiment, the drone 170 may be removably attached to the top of the eyeglass frames above the right lens/display 115 and left lens/display 120. In other embodiments, it may be attached elsewhere. Attachment of the drone 170 may be via magnets, clips, connectors, or similar means.

As shown in FIG. 8, the drone 170 may communicate directly with the eyeglasses 100 and/or via the smart device 180. When in direct communication with the eyeglasses 100, the wearer may control the drone 170 via voice commands received by microphone 165 and transmitted by transmitter 163. When controlled via the smart device 180, the downloaded App presents the displayed user interface including drone 170 control inputs (or voice commands). The remote control device 145 may also be used to control the drone 170. While the drone 170 may be formed as a portion of the eyeglasses 100, the drone 170 may also be a separate device linkable to the eyeglasses 100 via wireless technology.

As best seen in FIG. 1, the eyeglasses 100 may include a charging port 102 and data ports 103. The data ports 103 may be used to upload (e.g., software updates) and download data (e.g., sensor outputs, drone camera outputs, etc.) via wired connections using USB connectors and the like.

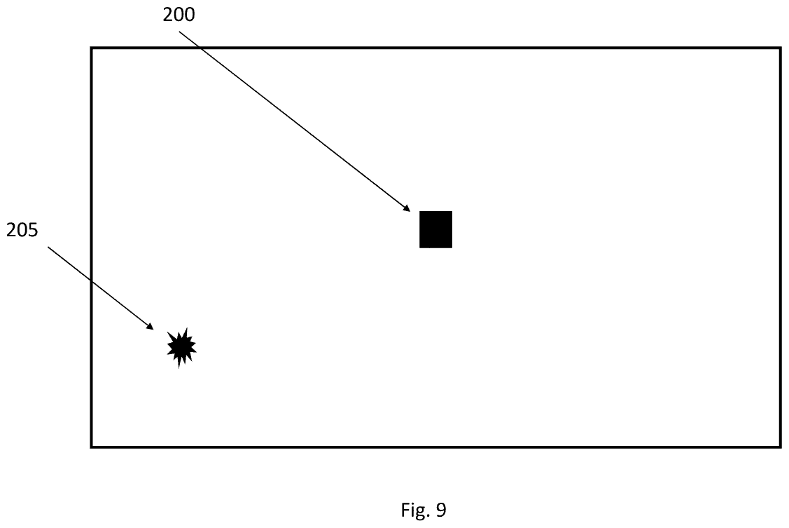

In one embodiment, the eyeglasses 100 may be utilized to conduct visual field tests. A visual field test is commonly conducted in an office setting and comprises sitting in front of a machine (called the Humphries Field Screener) and focusing on a small black centered square and pressing a button each time you see a flickering bar of light. The visual field test tests side or peripheral vision. The visual field test may not provide the best results since the test in conducted in a dark room whereas most people do not stay in dark rooms but spend a lot of time outdoors. Accordingly, the eyeglasses 100 may be used to conduct the visual field tests in an outdoor, natural setting. In such a case, as shown in FIG. 9, a black centered square 200 (or other symbol) and flickering bar (or other shape) 205 of light may be projected, displayed or otherwise depicted on each lens individually (as the other display or display area is blackened) as the wearer utilizes an input device to identify each time the wearer notices the flickering bar of light. Since the visual field test is conducted using the eyeglasses in a natural setting, the test results are more useful and accurate. Indeed, in one embodiment, the eyeglasses 100 may be configured/programmed to conduct the visual field tests routinely (e.g., every 3 months).

The data captured by the eyeglasses 100, may be uploaded and sent to third parties (e.g., ophthalmologist) for evaluation. The data may also be uploaded for short- and long-term storage.

Although the invention has been described in detail with reference to several embodiments, additional variations and modifications exist within the scope and spirit of the invention as described and defined in the following claims.

We claim:

1. A pair of eyeglasses comprising:
right and left temples;
one or more displays positioned in view of a wearer's eyes;
one or more sensors configured to determine the position of a wearer's eyes, namely pupils;
two or more cameras; and
one or more processors running software configured to (i) receive input in the form of video content from said one or more cameras and (ii) output manipulated video content to said one or more displays for viewing by a wearer thereof.

2. The pair of eyeglasses of claim 1 further comprising one or more gyroscopes.

3. The pair of eyeglasses of claim 1 wherein said one or more sensors are cameras, scanners or biometric readers.

4. The pair of eyeglasses of claim 1 comprising two separate displays with one display positioned in view of each eye of said wearer.

5. The pair of eyeglasses of claim 2 wherein said one or more gyroscopes are configured to output data associated with a position of a wearer's head.

6. The pair of eyeglasses of claim 1 further comprising a drone removably attached thereto.

7. The pair of eyeglasses of claim 1 further comprising one or more motors to change the distance between the one or more displays and the wearer's eyes.

8. An eyeglass system comprising:
a pair of eyeglasses including right and left temples;
one or more displays;
one or more sensors configured to determine the position of a wearer's eyes;
one or more cameras;
one or more processors running software configured to receive input in the form of video content from said one or more cameras and output manipulated video content to said one or more lenses/displays for viewing by a wearer thereof; and
a drone configured to be controlled via voice commands received and transmitted by said pair of eyeglasses.

9. The pair of eyeglasses of claim 8 further comprising one or more gyroscopes.

10. The pair of eyeglasses of claim 8 wherein said one or more sensors are cameras, scanners or biometric readers.

11. The pair of eyeglasses of claim 8 comprising two separate displays with one display positioned in view of each eye of said wearer.

12. The pair of eyeglasses of claim 9 wherein said one or more gyroscopes are configured to output data associated with a position of a wearer's head.

13. The pair of eyeglasses of claim 8 further comprising a drone removably attached thereto.

14. The pair of eyeglasses of claim 8 further comprising one or more motors to change the distance between the one or more displays and the wearer's eyes.

15. A pair of eyeglasses comprising:
right and left temples;
one or more displays positioned in view of a wearer's eyes;
a user input device; and
one or more processors running software configured to conduct a visual field test on said one or more displays by: (i) displaying a centered symbol on said one more displays; (ii) intermittingly displaying a flickering light on said one or more displays; and (iii) receiving inputs, via said user input device, by a wearer in response to observing said intermittingly displaying flickering light.

16. The pair of eyeglasses of claim 15 further comprising one or more gyroscopes.

17. The pair of eyeglasses of claim 15 wherein said one or more sensors are cameras, scanners or biometric readers.

18. The pair of eyeglasses of claim 17 wherein said one or more processors further run software configured to (i) receive input in the form of video content from said one or more cameras and (ii) output manipulated video content to said one or more displays for viewing by a wearer thereof.

19. The pair of eyeglasses of claim 15 further comprising one or more sensors comprising cameras, scanners or biometric readers.

20. The pair of eyeglasses of claim 15 further comprising one or more motors to change the distance between the one or more displays and the wearer's eyes.
